(19) United States
(12) Patent Application Publication
(10) Pub. No.: US 2006/0116767 A1
(43) Pub. Date: Jun. 1, 2006

(54) Implant Used in Procedures for Stiffening the Vertebral Column

(75) Inventors: Fritz Magerl, St. Gallen (CH); Roger Stadler, Zurich (CH)

Correspondence Address: VOLPE AND KOENIG, P.C., UNITED PLAZA, SUITE 1600, 30 SOUTH 17TH STREET, PHILADELPHIA, PA, 19103, US

(73) Assignee: Sepitec Foundation, Vaduz (LI)

(21) Appl. No.: 10/534,293

(22) PCT Filed: Nov. 14, 2003
(86) PCT No.: PCT/EP03/12762

§ 371 Date: May 9, 2005

(30) Foreign Application Priority Data
Nov. 14, 2002 (DE) 102 53 169.2
Nov. 14, 2002 (DE) 102 53 170.6

(51) Int. Cl. A61F 2/44

(52) U.S. Cl. 623/17.12; 623/17.13

(57) ABSTRACT

The invention concerns an implant in the form of a compression resistant hollow body (1), used in procedures for stiffening the vertebral column (2). The hollow body (1) includes two open receptacles (3, 4) mutually oriented toward one another that interlock and form the implant proper (cage). The two receptacles (3, 4) can be separated by pressure through insertion of a filling material, thereby producing an expansion of the hollow body (1) after it has been implanted.

### BACKGROUND

The invention concerns an implant used in procedures for stiffening the vertebral column.

Medical technology refers to such an implant as a Cage, which, in procedures for stiffening the vertebral column is inserted in the intervertebral space, after removing the intervertebral disc. Bone or bone substitutes taken from the patient are fixed in the implant. In addition to hollow cages there are also a few full cages.

These implants are inserted from the front through an anterior lumbar interbody fusion (ALIF). Usually two Cages are used for the technology using a posterior lumbar interbody fusion (PLIF); that is one from each side (bipolar technology).

Conservative treatments with uncontrolled and excessive pain caused by instability, spinal cord and nerve root compressions, deferred positions and tumors may form indications for intercorporal spondylodesis. During the surgical procedure the causes for pain and pathological changes are removed and the spinal column stability is restored using a spondylodesis.

In an intercorporal spondylodesis the intervertebral disk is always removed. Often vertebral bodies also require total or partial removal. In either event the load of the vertebral column may be restored. This can be performed, for example using pressure resistant bone grafts from the patient that are then pinned between the vertebral bodies. However, the load capacity of such grafts is often uncertain. Since the availability of suitable bone grafts is limited and significantly increases morbidity,

pressure resistant implants manufactured from metal or other material are used increasingly instead of the autogenous bone grafts. Technical terminology refers to this implant as a Cage.

The stability of the spinal column can definitely be restored, since at least two invertebral discs are removed during an intercorporal spondylodesis. For example this can be done using pressure resistant bone grafts removed from the patient. Since the load capacity of such grafts is often uncertain and its availability is limited and, additionally, the removal of grafts causes an increase in morbidity, instead of autogenous bone grafts, bone substitute material is increasingly used, as well as pressure resistant intervertebral implants (also called Cages).

Intervertebral implants can be inserted from the anterior (ALIF=anterior lumbar interbody fusion) into all areas of the spinal column. The implants can also be inserted in the lumbar portion of the spine from the posterior (PLIF=posterior lumbar interbody fusion), the side or the posterior side. One or two implants are inserted for each intevertebral disc space. Biportal implantation is called the insertion of two implants from the posterior through two separate openings of the intevertebral disc ring. In a uniportal implantation the intervertebral disc ring is only opened at one location.

An intervertebral implant functions as a pressure carrying place holder, which stabilizes the spondylodesis, secures the alignment to the vertebral bodies and ensures that a solid bone bridge is formed between the vertebral bodies. Bone or bone substitute material filled into and/or around the implant provide the matrix for the bone regeneration. The stability of the spondylodesis plays a deciding role for the ossification process. Movements occurring within the spondylodesis delay or prevent osseous consolidation. Pedicle systems or translaminar screws are used to additionally stabilize the spondylodesis.

The expansion (disktraction) has mechanical significance in addition to the therapeutic significance: the associated stretching of soft tissue associated generates a force which will fixate the intervertebral implants between the vertebral bodies. This prevents movements within the spondylodesis and also avoids the risk of a sometimes momentous secondary implant dislocation.

During the implantation of intervertebral implants it is often very difficult, especially in minimal invasive and uniportal implantation technology, to achieve effective expansion.

Cages can bridge intervertebral disk spaces or replace additional portions of vertebral bodies, as well as entire vertebral bodies. Cages can be implanted in all areas of the spinal column either anterior (ALIF), at the lumbar portion of the spine and also from the posterior (PLIF). In a spondylodesis bone or bone substitute material is inserted between two or more vertebral bodies (intercorporal spondylodesis) or fixated through the back vertebrae elements onto two or more vertebrae (dorsal spondylodesis). During the course of several months a bone bridge develops which fuses the vertebrae and thus eliminates the instability causing the pain. A condition for the bone healing to the vertebrae is that the affected section of the spinal column must be immobilized until the bone bridge is fixated adequately.

The scope of the spondylodesis includes normalizing the reduced vertical distances caused by the invertebral disc narrowing between the vertebral bodies and removal of the structural aberrance of the spinal column. The expansion is always an integral part of the procedure, since alone widening the vertical vertebral body distance by the “expansion” effected instrumentally, will have a significant decompressing effect on the nerve structures, as well as on the intervertebral joints.

The purpose of a cage is to act as a pressure carrying placeholder, which ensures that the vertebral bodies will fuse at the desired location. The latter occurs by newly developed bones that produce a bridge between the vertebral bodies. Bone or bone substitute material is the matrix for the bone building that is filled into and/or around the cages. Depending on several factors this ossification process requires several months. The stability of the spondylodesis plays a deciding role here, since the repetitive movements between the cage and the vertebral bodies can significantly delay or prevent the bone building of the spondylodesis. Spondylodesis is the surgical stiffening of a section of the spinal column. With conservative treatment methods, uncontrolled and excessive pain, spinal cord and nerve root compressions and deferred positions are all indications for spondylodesis. Pain may originate from all abnormally changed structures of the spinal cord. In many cases they are based on the ‘instability’ of movement segments, caused by degenerative changes of the intervertebral disc and intervertebral joints of the movement segment. The narrowing of the vertebral canal or the vertebral foramen is usually responsible for spinal cord or nerve root compressions. A surgical procedure may remove the pathological changes and restore the stability of the spinal column through a spondylodesis.

A significant challenge of the surgical technology is to normalize the reduced distances between the vertebral bodies or to eliminate the vertebrae shifts. The widening of the intervetebral distance has in addition to the therapeutic significance a not to be neglected mechanical significance: the strain associated with pushing apart the paravertebral soft parts generates a counter force which presses the vertebral bodies onto the cage, which is wedged between the vertebral bodies. This prevents damaging movements between the cage and the vertebral bodies and reduces the risk of a sometimes momentous secondary cage dislocation.

The purpose of this invention is to simplify the cage implantation technology and thus to reduce the surgical trauma by applying less invasive surgical techniques, which can be applied more often as a result of this simplification.

### SUMMARY

In accordance with the invention this can be accomplished as follows: the implant is composed of a perimeter enclosed hollow body made up of at least two box type open moveable receptacles, mutually oriented which interlock and which are telescoping, which by inserting filling material or by using a filling made of elastomer can be separated by pressure in order to effect an expansion of the hollow body.

Thus an expandable hollow body (Cage) has been created, which is a relatively small dimension when in a pushed together condition of the receptacle, is easily inserted in the intervertebral fissure or other defect in the vertebral body column, where it is then expanded to its required size. This also allows the one sided use from the back (uniportal PLIF). Additionally, instead of using the standard two cages only one can be implanted. The usually in advance performed spreading apart process is then completed by the hollow body.

The invention also provides an elastic intervertebral implant to promote the bone fusion of the spondylodesis, in which the pressure differences appearing in the contact zone of the bones may be reduced and the bone rebuilding can be stimulated by the elasticity or reduction of the “stress shieldings”. The implant will also affect an expansion of the vertical vertebral body distance.

The difference between elasticity of the bone and the implant can play a significant role when using intervertebral implants. The bone of the vertebral body is elastically more deformable than implants manufactured from metal or polymer. Through the daily load changes, stress peaks may appear in the contact zone of the bone, which can cause micro fractures in the bone of the contact zone, due to material wear. The damaged bone is then broken down and replaced by connective tissue. With an x-ray this replacement can be seen well by the fairly wide line of lucency surrounding the cage. Replacing the fixed bone using soft connective tissue leads to a loosening of the cage (implant) or instability of the spondylodesis. As a consequence of the instability the development of a bone bridge between the vertebral bodies may not happen, which means that a pseudarthrose may be created. This is synonymous with a failure of the treatment.

The process is strengthened if the spondylodesis is not quite stable a priori that is, if movements are possible right from the start between the vertebral bodies and the implant. If, for example the vertebral body can move slightly away from the implant when leaning back, then the load on the bone drops to zero. Since in such a case the loads can each increase from zero to a maximum, the wear fracture responsible load differences are especially high.

The present invention produces an elastic hollow body used to restore the spinal column stability, which is implanted as part of an intercoporal spondylodesis, between the vertebral bodies in the vertebral disc space or in the defect vertebral body column. The hollow body is formed by two open receptacles which interlock and are filled with a tissue compatible elastomer. High stress changes on the implant that may occur during a cyclical load in the contact zone of the bone that may lead there to bone degradation and a subsequent instability, can be avoided by approximating the elasticity of the implant to the bone. The ossification of the bone transplant by approximating the elasticity of the implant to the bone is promoted and thus ensures the success of an intercoporal spondylodesis.

A simple design is presented if two interlocked open receptacles are provided. Merely two parts, fitting exactly into each other are used. In accordance with the invention, the hollow body (cage) is designed in a manner that it also suits the bio-mechanical requirement of the implantation of only one cage. This, combined with the telescoping principle significantly simplifies the cage implantation technology and especially the minimal invasive and uniportal implantation technology.

Furthermore, an advantage is that the receptacle can be fastened to a supply hose. Thus, already upon inserting the receptacle a proper supply of fill material can be ensured. Thus access is only necessary from one side (just the back).

To enable a simple and uncomplicated supply of the filling material, it is recommended that the other end of the supply hose be connected to a device producing the necessary filling pressure.

The coupling hole for the supply hose can simultaneously be used to feed in the hollow body, to create a secure position during the implantation. For this purpose it is recommended that the opening planned for connecting the supply hose has an attachment used to feed in the hollow body.

It is especially beneficial if the filling material is made of a tissue compatible material that stays liquid or, after a liquid phase, is made of self hardening material. Therefore, the pressure necessary for the expansion of the receptacle can be administrated exactly with the material that presents the best solution for the particular application.

To affect a better placement and a better integration of the implanted hollow body in the bone structure, it is envisioned that the hollow body is structured or coated on a portion or on its entire surface.

If it is further envisioned that the receptacles forming the hollow bodies are sealed with one another to effectively be prevent the filling material from escaping from the receptacles.

It is further provided that the receptacles forming the hollow bodies are adjustable relative to each other; however the adjustment movement is limited to an area that always provides for a mutual overlapping of the receptacles. This will also ensure that the filling material does not escape the receptacles unintentionally.

Further, it is beneficial if the adjustment movement between the two receptacles is limited by screws that catch in a slit. This prevents the receptacles from being pushed too far away from each other.

In another embodiment of the invention, it is envisioned that the elastomer is filled in the interior portion of the hollow body. The elastomer's only function is to form the desired elasticity, thus the elastomer will never directly come into contact with the surrounding area of the implant.

The design plans here that the elastomer completely or partially fills the hollow body. As a result other version options are created, among others also using different areas of different elasticity.

Preferably the elastomer should completely fill the interior room of the implant to avoid squeezing gas or liquid out of the implant or into the implant upon loading or unloading, respectively.

In connection with this, it is also possible that the elastomer filled into the hollow body is loosely or tightly attached to make a seal on the interior side walls of the hollow boy.

If it is further provided that the interior surfaces of the upper and lower wall of the two interlocking receptacles of the hollow body are penetrated during loading of the filled-in elastomer, then the most varied load distribution and load emergence in the implanted condition of the implant can be specially addressed.

In another embodiment of the invention, a cavity is provided below the filled-in elastomer, which is between the elastomer and the lower wall of the interlocked receptacles of the hollow body.

This will increase the elasticity of the implant even further, or additional agents can be inserted to change or increase the elasticity.

One embodiment of the invention provides for incorporating in the elastomer an air tight air bubble. This signifies a very simple and constructively very effective design to increase elasticity.

In one particular embodiment, a device, for example in the form of a clamping screw, is attached to the hollow body to allow the hollow body to be compressed to minimum height before the implantation, and to be expanded after the implantation. This will facilitate the implantation and lead to augmenting the vertical vertebral body distance expansion. Additionally, the intevertebral implant will have an initial tension, which will keep the implant in constant contact with the vertebral body and reduce the damaging tension differences.

As an optimal option of inserting the implant, an embodiment is provided where the exterior receptacles of the hollow body have a wedge-shaped insertion end. This will significantly improve the expanding effect.

In a constructively simple form of the invention, depending on the form to be chosen and depending on the purpose, the implant is manufactured using metal, polymer or composite materials.

It is further recommended that during the manufacture using polymer or composite material, elements or materials are incorporated in the implants that give off radiological shadows. This will make the implants radiological visible.

Various constructions of the implant according to the invention are possible in different ways. However, a design is most beneficial if the pressure transducing parts of the hollow body take the form of a low cylinder or prism that includes cover plates that are equal or slightly vaulted, parallel or slightly pitched against each other.

It is preferred that the implant is designed to be attachable to am implantation instrument to simplify the implantation process. This will guarantee that the implant itself ensures for a good grasp and insertion.

A beneficial further embodiment provides that the surface of the implant is structured and/or coated. This will ensure a safe positioning of the implant at the implant location and will provide a better integration into the bone implant bed.

### BRIEF DESCRIPTION OF THE DRAWINGS

Other features of the inventions and further benefits are detailed in the following description with the help of the drawings. In the drawings:

FIG. 1 a section view through a section of a spinal column, whereby insertion of the implant in the form of an expandable hollow body (cage) can be seen;

FIG. 2 a view of the pressure secure implant according to the invention;

FIG. 3 a side view of the implant seen from the same side as in FIG. 2, whereby the two receptacles of the implant designed as a hollow body are presented uncompressed;

FIG. 4 a view of the implant from the top, whereby a connection element used as a supply conduit is presented as in FIG. 3;

FIG. 5 a perspective view of the implant with an attached insertion instrument;

FIG. 6 a perspective view of an embodiment of the implant including a wedge-shaped insertion end

FIG. 7 a section view of the invertebral disc space including an inserted implant of an alternate design

FIG. 8 a section of the implant inserted as defined in FIG. 7, in simplified form;

FIG. 9 and FIG. 10 sectional views through an implant in simplified presentation, whereby the compressed position is shown (FIG. 9) and the expanded position is shown (FIG. 10);

FIG. 11 and FIG. 12 sectional views through the implant in simplified form, whereby the compressed position (FIG. 11) and the expanded position (FIG. 12) are shown and each includes a gas bubble as an additional elastic element; and

FIG. 13 a perspective view of another embodiment of the implant.

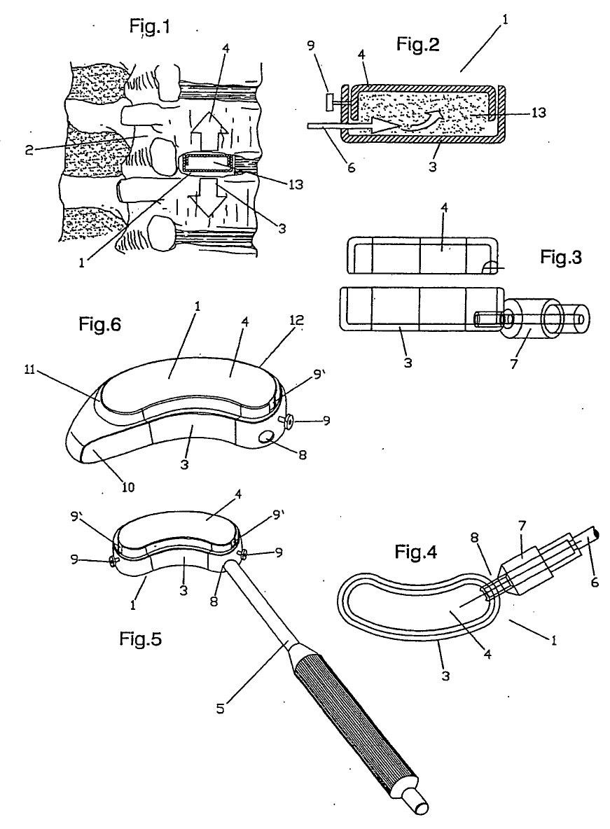

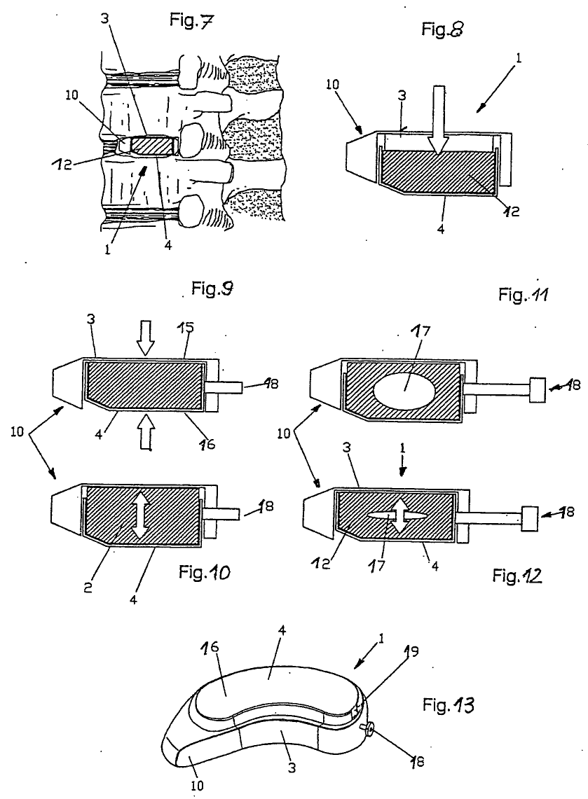

### DETAILED DESCRIPTION OF THE PREFERRED EMBODIMENTS

An implant in the form of a pressure resistant hollow body 1 for the use in procedures for stiffening the spinal column 2 is provided which, after removal of the vertebral disc(s) and/or after partial or total ectomy of vertebral bodies, can be inserted from the ventral or dorsal into the created defect, and is expanded to the required size at the implanted location. This hollow body 1 is produced from at least two open, interlocking, mutually oriented receptacles 3 and 4, which fit perfectly into each other, so that also an applicable pressure build-up in the hollow body can allow achieving the necessary expansion pressure. The pressure build-up is achieved by inserting filling material in the interior space 13, whereby the two receptacles 3 and 4 are pushed apart and thus affect an expansion of the hollow body 1 in a vertical direction.

The hollow body 1 is inserted in the compressed condition of the two receptacles 3 and 4, possibly with the help of instrument 5 attached to the cage supply opening. In a compressed condition the cage has relatively small dimensions. After insertion, the filling material obtained from a container equipped with a device to produce pressure, is squeezed into the hollow body 1 with the necessary pressure, using a detachable supply hose 6 or another supply conduit. The now expanded hollow body 1 will thus completely fill out the defect, expand the distance between the vertebral bodies and will produce the counter pressure necessary for the wedging of the cage. The filling material must be faultlessly tissue compatible and viscous enough that it can flow through the supply hose 6, but not escape the cage.

Filling material can be a tissue compatible, liquid medium or a tissue compatible, cold hardening material after the liquid phase insertion. In the event of a liquid medium, a return valve needs to be attached to the cage, which will avoid the filling material from flowing back and a sealing screw is necessary that is used to lock the supply opening to the cage. In the event of using a self hardening material, the supply hose is removed after the polymer has hardened in the cage.

A portion of the bone or bone substitute material is attached to the defect before inserting the cage in a manger that it will finally come to rest before the cage. The other portion will be stuffed next to and behind the cage after removing the supply hose. This ensures that an adequately strong bone bridge can develop around the cage.

At least two interlocking receptacles 3 and 4 are used to build the hollow body 1. In this case two open, interlocking receptacles are used. However, the invention also allows on the perimeter of hollow body 1 to push out more than two receptacles similar to pushing a set of drawers. When building up pressure in the interior of the hollow body 1, expansion movements can thus be triggered into several directions. Another version may be obtained by using several receptacles 4 that are inserted in one or several openings in a receptacle 3. Here it is also possible that a separate supply hose exists for each receptacle 3, so that different pressure can be build up for different areas.

The supply hose 6 can be attached in different ways to the hollow body 1. For example it is possible to mount it or screw it onto a connection piece 7 of the cage. A type of bayonet catch or direct mounting or screwing to the connection of the cage is also possible. When using a hardened material it must be made sure that the supply hose 6 can be removed after the material has hardened.

The opening 8 of the cage used for fastening the supply hose 6 can also be used to attach instrument 5, which is suitable for inserting the cage to the defect.

The hollow body can be formed from a variety of material: metal, polymer or a composite materials. It is also possible to manufacture the cage from a bone substitute material or from a self-dissolving material, which can finally be replaced by bone. The latter assumes that a tissue compatible and absorbable medium was used as the filling material.

The surface of the receptacle can be fully or partially structured or coated, to promote the adhesion of the bone to the cage.

To avoid that the overlapping areas of the cage halves become too small during the expansion of the cage, a device is provided which will prevent the cage halves from being pressed apart too much. This device can for example comprise a peg or screw 9, which is attached to the exterior portion of the cage and catches in a nut 9 of the interior receptacle 4. One or several seal rings, in the form of piston linings, running parallel to the end plates of the cage may also be used which, when reaching the maximum allowed expansion, hook to a border or other elements of the interior area of the exterior receptacle 3.

The most significant features of the inventions are especially the design of a telescopic vertical, possibly also radial expandable cage comprising at least two receptacles to be used to stabilize the vertebral body column. The force necessary for the expansion is generated by the filling material squeezed into the hollow body (cage). The filling material is squeezed into the cage through a device generating pressure that is attached to supply hose attached to the cage. Filling material can be a tissue compatible liquid, as well as self-hardening material after the liquid phase. To reduce the risk of infection, suitable antibiotics can be added to the filling material. The receptacles (cage parts) must interlock exactly or be sealed in a manner that no filling material can escape. Devices are planned that limit the expandability of the cage in a manner that the contact areas of the cage receptacles are always large enough to especially prevent totally pushing the receptacles apart from each other.

An instrument 5 can also be attached to the opening used for the filling delivery, to insert the cage into the defect. The surfaces of the cage receptacles may be structured or coated, to facilitate bone growth onto the cage.

As stated, according to the invention, after filling, the cage does not contain any dead space where bacteria could accumulate. The cage can be implanted from the ventral or dorsal sides into the defect located in the area of the spinal column. One or more cages can be used uniportal or biportal for stabilizing the spinal column, depending on the situation and the requirements.

The cage (the hollow body) has the following most significant devices and features: the implant is in cylinder form or bean shaped (see especially FIGS. 4 to 6). The upper and lower area of the receptacle 3 and 4 are slightly arched towards the direction of the longer and shorter diameter. The ends positioned in the longer diameter of the parts are each equally high. In contrast the front-wall of the part is a little lower than the back wall. The design illustrated in FIG. 6 shows that a supplemented constructive design can be equipped with a wedge shaped insertion part 10 arranged at the end of receptacle 3. An expanded effect can be reached in this space already upon insertion into the vertebral disc space. The part of the implant situated across from the insertion part 10 is rounded and shows the boring 8 for taking up instrument 5, which is used for the implantation of the cage.

The following devices are planned to facilitate turning the implant into the definite diagonal position: as of the passage of the insertion part 10 into the receptacle 3, the upper and lower leading edges 11 of the implant are sharp edged. The leading edges 12 are gently rounded off at the implant end positioned across from the insertion part 11.

The sharp edges will cut into the cover plates upon inserting the implant from the posterior or side into the vertebral disc space. Consequently, the implant will already begin to turn into the desired diagonal position upon insertion. The implant can be additionally guided at the beginning through rod-shaped insertion instrument 5 attached at its back end. The instrument 5 is removed as soon as it has reached the boundary of the insertion opening and can not be swiveled any further. Thereafter, the implant is hammered in further using a ram, which is attached at the back end and the implant is simultaneously turned into its final position.

In principle such implants can be inserted in all areas of the spinal column: at the cervical, thoracic and lumbar spine from the front and at the lumbar spine also from the back uniportal or biportal, from the side or sideways from the back. The condition for applicability is that the form of both receptacles is specially designed in accordance with the anatomical factors of the planned implantation location (region of the spinal column) and the planned implantation technology.

In the embodiment illustrated in FIGS. 7 to 13, the implant is a hollow body 1 filled with elastomer 12, whereby the hollow body 1 includes at least two interlocking, movable receptacles 3 and 4, mutually telescoping in the body longitudinal axis direction. The elastomer 12 is filled into the inner receptacle, whereby the elastomer 12 completely or partially fills the hollow body 1. The elastomer 12 is positioned loosely or firmly and sealed to the inner side walls of the receptacle 4 of the hollow body 1.

The inner surface of the upper wall 16 and the bottom wall 15 of the receptacles 3 and 4 of the implant are designed in a manner that they can penetrate into the elastomer 12 if pressured. To increase elasticity a hollow space can be left beneath the elastomer 12, which is between elastomer 12 and the bottom wall 15 of the implant, or, as shown in the embodiment of FIGS. 11 and 12, an air bubble is incorporated in the elastomer 12.

A device is attached to the implant, for example in the form of a clamping screw 18, which allows the implant to be compressed before implantation to minimum size, so that it can then be expanded only after implantation, after the clamping screw 18 has been loosened. To prevent the area of expansion from going too far, the end of the clamping screw 18 will catch in slit 19 (see FIG. 13) to limit to a certain degree receptacle 4 from pushing out of receptacle 3 of the hollow body 1 too far.

The exterior receptacle 3 of the implant may have a wedge shaped insertion part 10 used to increase the stated expansion effect.

From a functional view, such an intervertebral implant with wedge shaped ends is comprised of two parts. FIG. 8 shows an embodiment. The two parts are the receptacle 3 including the wedge shaped insertion part 10 and the receptacle 4 of the implant. The form of the two receptacles depends upon the area of the spinal column the implant will be inserted and which technology will be used.

The receptacles have the following base forms: it is a blunt wedge shaped insertion part 10, which can be attached on any side of receptacle 3 and is used to insert the implant into the vertebral disc space. The upper and bottom areas of the insertion part 10 are even and slanted so that the wedge height decreases from the wedge base to the wedge end. The end of the wedge positioned across the wedge basis is vertically rounded off in the side view and in the oversight.

The pressure absorbing receptacles 3 and 4 may have the form of a low cylinder or prism, including even or slightly arched, positioned parallel or slightly opposing base and cover plates.

The implants may contain a device used to attach an implantation instrument. The surfaces of the implants may be structured and/or coated.

The elastic intervertebral implants are made of metal, polymer or composite material. Elements or material providing radiological shadows are incorporated in the implants, in order to make the position of invisible implants manufactured from polymer or composite material visible in an x-ray.

Within the scope of the invention it is possible to form the hollow body 1 from more than two open receptacles 3 and 4 that are mutually oriented. Thus, it would be feasible to, for example form receptacle 4 from several partitions that are independent, flexible and relatively moveable to receptacle 3. It would then also be possible to completely or partially fill the different partitions with elastomers having different elasticity.

The following implant technology is possible with this implant defined in the invention: after a possible prior expansion of the vertebral disc space or the defect using a suitable instrument (e.g. forceps), the height of the vertical vertebral body distance is measured and the implant is chosen that in a compressed (lowest height) condition can be inserted into the vertebral disc space. The implant is squeezed or hammered into the vertebral disc space, possibly with the help of an implantation instrument attached to it. After the final positioning the clamping screw 18 is loosened so that the implant can be expanded into a vertical direction.

The implants in a uniportal dorsal or dorsal lateral application technology must be turned during the implantation from the initial sagittal or sloped implantation direction into the frontal direction, especially if only one implant is inserted into the vertebral disc space. During the desired locking of the implant the rotation can present significant difficulties or be impossible.

The uniportal intervertebral implant has the following significant devices and features: the implant is bean shaped (see especially FIG. 16). The upper and bottom area of receptacles 3 and 4 are slightly arched in the direction of the longer and shorter diameters. The ends of the receptacles positioned at the longer diameter are each of equal height. In contrast the front wall of the receptacle is slightly lower than the back wall. The section of the implant positioned across from the insertion part 10 is rounded off and may included a device used to accommodate an instrument, which can be used for clamping and implantation.

To facilitate the rotation of the implant the following devices are planned: from the transition point of the insertion part 10 into the receptacle 3 the upper and lower front edges of the implant are sharp edged. As of the implant end positioned across from the insertion part 10 the front edges become more rounded. On the upper and lower surface of the receptacle 3 notches are placed against part 10 parallel to the front edges; their back areas are positioned horizontally to the respective surface and their front area is flat against the surface of the implant. The notches work as guide grooves.

Already when inserting the implant from the back or back side into the vertebral disc space the sharp edges and the guide grooves will cut into the cover plates. Thus, the implant will already begin during insertion to rotate into the desired direction. Through a rod shaped insertion instrument attached to the back end, which simultaneously will function as a clamping screw 18, the implant can be guided additionally at the beginning. The instrument is removed as soon as it has reached the insertion opening and can no longer be pivoted. Thereafter the implant is hammered further in using a ram attached to the back-end, and is, at the same time, rotated into its final position.

Such intervertebral implants can usually be inserted in all areas of the spinal column: at the cervical, thoracic and lumbar spine from the front and at the lumbar spine additionally uniportal or biportal from the back, from the side or from the back side. The condition for application is that the form of both implant parts is specially designed to meet the anatomical factors of the planned implantation location (region of the spinal column) and the planned implantation technology.

Instead of the air bubble 17 developed in elastomer 12 or, in addition to such a design, another spring element could be inserted in the hollow space between the bottom wall 15 of receptacle 3 and the inserted receptacle 4 filled with elastomer 12, in order to increase the elasticity of the entire implant. Here for example the use of a spring element in the form of a spiral, screw or flat spring would be possible, or another inlay in the form of an elastomer would be possible, which would have different elasticity in comparison to the elastomer 12 contained in the receptacle 4.

Since the elastomer 12 works like a spring it would certainly be possible to consider it to fall under the term elastomer and to illustrate this elastomer as a spring, so that instead of a filling made up of elastomer filling material, one or several spiral, screw or flat spring(s) could be utilized.

1. Implant used in procedures for stiffening the vertebral column, the implant comprising an enclosed hollow body which includes at least two movable open receptacles (3, 4), oriented toward one another which interlock, and can be spread apart by inserting a filling material or by utilizing a filling material made of an elastomer (12) in order to expand the hollow body (1).

2. Implant according to claim 1, wherein there are two of the open receptacles (3, 4) which interlock.

3. Implant according to claim 1, wherein the implant can be connected to a supply hose (6).

4. Implant according to claim 3, wherein an other end of the supply hose (6) is adapted for connection to a device used to generate a filling pressure.

5. Implant according to claim 3, wherein an opening (8) for connecting the supply hose (6) is also used for attaching an instrument (5) used to insert the hollow body (1).

6. Implant according to claim 1, wherein the filling material is made of a tissue compatible, liquid or initially liquid phase, self hardening material.

7. Implant according to claim 1, wherein the hollow body is structured or coated on one part or over an entire surface thereof.

8. Implant according to claim 1, wherein the receptacles (3,4) forming the hollow body are sealed with one another.

9. Implant according to claim 1, wherein the receptacles (3,4) forming the hollow body are adjustable relative to each other, whereby adjusting movement is limited to a certain area, which ensures a mutual overlapping of the receptacles (3,4).

10. Implant according to claim 9, wherein the adjusting movement between the two receptacles (3, 4) is limited through a screw (9) in one of the two receptacles catching in a slit (10) in the other of the two receptacles.

11. Implant according to claim 1, wherein the elastomer (12) is filled into an inner portion of the hollow body (1).

12. Implant according to claim 11, wherein the elastomer (12) completely or partially fills the hollow body (1).

13. Implant according claim 11, wherein the elastomer (12) filled into the hollow body (1) is loosely or firmly fitted to an inner side wall of the hollow body (1).

14. Implant according to claim 1, wherein inner surfaces of upper and bottom wall (16, 15) of the interlocking receptacles (3,4) of the hollow body (1) penetrate into the filled in elastomer (12) when compressed.

15. Implant according to claim 1, wherein a hollow space is left below the filled-in elastomer (12), which is between the elastomer (12) and a bottom wall (15) of the interlocked receptacles (3,4) of the hollow body (1).

16. Implant according to claim 1, wherein an airtight air bubble (17) is incorporated in the elastomer (12).

17. Implant according to claim 1, wherein the hollow body is compressed to minimum height before implantation and a device, such as a clamping screw (18), is attached to the hollow body (1) to expand the hollow body (1) after implantation.

18. Implant according to claim 1, wherein an exterior one of the receptacles (3) of the hollow body (1) has a wedge shaped insertion end (10).

19. Implant according to claim 1, wherein the implant is manufactured from metal, polymer or a composite material.

20. Implant according to claim 19, wherein in manufacture using polymer or composite material, elements or material are incorporated in the implant that produce radiological shadows.

21. Implant according to claim 1, wherein the receptacles (3, 4) of the hollow body (1) can be pressurized and have a form of a partial cylinder or prism, whereby base and cover plates are included that are even or slightly arched and are positioned parallel or slightly slanted relative to each other.

22. Implant according to claim 1, wherein the implant includes a connection for attaching an implantation instrument.

23. Implant according to claim 1, wherein a surface of the implant is structured and/or coated.
